SEQUENCE LISTING (26 sequences)

SEQ ID NO: 1 - DNA, 191 bp, artificial sequence
  ESB marker
ggtgtggaggtgttcaaaggcattgacaatcggactcagaaagtagtcgccataaaaatcattgacctggaggaggcagaagatgagatcgaggacattcagcaggaaatcacagtgctgagtcagtgtgacagtccctacgtaaccaaatattacggatcctacctgaaggacactaaattgtggataat

SEQ ID NO: 2 - DNA, 244 bp, artificial sequence
  ESB marker
tatctgcagaatttccccttgagaagcgttatggggtgcaggtaagttattacacaagagaaagaagttttcttactaacagcaagattaatggcacaattcaaccaaaactcatatacattttactgcttaatttacatattattttggtggaaaaaatagtattctttattctttcagtttctttatgcaaaaatacacttctacagggacatcacttagatgttatgcaaacctccccccc

SEQ ID NO: 3 - DNA, 325 bp, artificial sequence
  ESB marker
gagacatttggccaaaagaggaatttccaggacaccaacaacatccattattccattattcatttgtttcctgaagagcaaacacttccttgaaattcttctcaaattctgcctccagtctaagccccatttggccaaaatcattgaacttgaaagatgccctgtggttctgaaagatgagacgcatgtcccacacaaacccttccacattggagtagccctgctcattcagcctcttcttgatcttgtccagccacatgggctccttgaggtttttagaagcctctttcatataataataatagggaatcctcactataacgct

SEQ ID NO: 4 - DNA, 688 bp, artificial sequence
  ESB marker
aagcgttatgcaggtaggccgacaaggcgaagtgggatgccggagagcggccgagttattgctccgaggagaccacgttcaccggttactatggcgaccgccccatcccggatcactatcagccgttcaccgccgatgaggcgacgtggttccagctctgggagacggtgagcgagggcactcctacgtcgccgcccttcgcgacgattgaggaactggcagcctacctcgccgagtggggcgacttctgtgatcacaggcgcgccgtcgagtccatggacgcgcgcgagattgagcgcctcctgacgctgaatgaccggcactagttcaaggtgcggctgggggcagcagcgcgcctaagctttctgcaagactggctgggcgcccagcatgatggtccgcggcggcgagatcctgaccaaccctggggacatggtgtcgtcgtgaccctcgcctagctctctcacacacctaggaggaagagatgaccacccccaacattcgcggccacgagaccgaagccaaggcccgcaaggcggcgatgaagtggttcaccttcacggacggcaccaagcctgtcgagggcgtccacttccacatcaagcagaaccacttcgggctctggaccttccgggagggcccggctccgaagtccgccggaccccgcatcactcataacgcttctcaa

SEQ ID NO: 5 - DNA, 373 bp, artificial sequence
  ESB marker
agaagcgttattgctgatacccgctacatgttctccaggcctttcagaaaacatggagttgttcctttggccacatacatgcgaatctacaggaagggtgatattgtagatatcaagggaatgggtactgttcaaaaaggaatgccccacaaatgttaccatggcaaaactggaagagtctataatgtcacccagcatgctgttggcatcattgtaaacaaacaagttaagggcaagattcttgccaagagaattaatgtgcgtatcgagcatattaagcactctaagagccgagatagcttcctgaaacgtgtgaaggaaaatgatcagaaaaagagggaagccaaagagaaagggacttggggttaacacc

SEQ ID NO: 6 - DNA, 235 bp, artificial sequence
  ESB marker
gggcggaggtcaccctggggatcctccagggccaggccctggcacaactcgtctccatcacacagatgggccgtcgcctggtcgtggctctcaggagtcagaccggaaaaagccagccctggggcaaccaggagcaccgaggtgatgagcaggacagcccaggaggtcatgttgaggcagctgaaaggtctgtgcaagtcaatcatgaagaaatttctccgtaccatcacctccc

SEQ ID NO: 7 - DNA, 285 bp, artificial sequence
  ESB marker
cttgtgtaggcagaggttccagggtcagtggaggaagcagcatcacagccagatccatggttgggggatggccacgggaaatgacttggtgactgactctgatctcagagtgggacaggctgacaggcatctgggaattccgggcaaggtcaggcacgtattatagaagagcaaacaccaatcccaaaatatcctcaggaatcagcgcatgagccccttctggctcctgtgatggatgatgaggcccagcccaaggaagatcagccccagcacaaagcctccaac

SEQ ID NO: 8 - DNA, 235 bp, artificial sequence
  ESB marker
  misc_feature (44)..(85): N = A, T, G or C
tctgcagaattcgcctctgagaagcgttatccgttggacccaannnnnnnnnnnnnnnnnnnnnnnnnnnnnnnnnnnnnnnnnncaagagaaaagatcagagggtgctggtgtgacgtttaagtaggaaaaggcctggaaggtgagtccatcaaccgcggagacaaaagtgggcccggctccttccacaggtgccgactgatgctgccagttcacggtcagtgtgggtcaacac

SEQ ID NO: 9 - DNA, 400 bp, artificial sequence
  ESB marker
  misc_feature (18)..(57): N = A, T, C or G
aagcgttatttagataannnnnnnnnnnnnnnnnnnnnnnnnnnnnnnnnnnnnnnntacacccagagtattccatagtttgatggttttgtctcgggagccagagacaatttgccggttgtcagaagagaaggccacactcagcacatctttggtatggcctacaaatcggcgagtggtggtgcccgttgtgagatcccaaaggcgaagggttccatcccaggagcctgagagggcaaattggccatctgaggaaatgaccacatcactaacaaagtgggagtgaccccgaagagcacgctgtgggataccatagttggtttcatctctggtcagcttccacataatgatggtcttatctcgagaggcggacgatatcatgtccgggaactggggagtg

SEQ ID NO: 10 - DNA, 397 bp, artificial sequence
  ESB marker
gggccaggggatgatatgaatgtcacaggaggagacaccttctgtctttgtttcaaagaaagttgatgtgccatttgttaatatacaagagaaatattgaaaatatattgaaaagagcaattttaaattatttttggcttatgttgcaatatttattttcttgtattaggaaagattcctttgtagaaaaaaaatgtatttttcattaacgcaaaaacctatttctcctttttgtacattgtccatgttcgctacccttaacgagcaatagaatgtatggctgcctcggggtggccggtgcccgcgtgccctgcatgattctgtggtcccaccaccatgtagctcccagtcccatcctgtcctgctcactcatgggggtttccagagcctagcccct

SEQ ID NO: 11 - DNA, 397 bp, artificial sequence
  ESB marker
tggattgcaggtgactgagaaaaccatcgaggacagtttttaaggggtcactgagccaggagcaaatgagatcctgagaaagtacttcattgtggaagagttagcactaagcaggaaacctttccatgctgtgaagaagctgggacagaaggttcttccttgagtgtgaccatcttcacttcagctcaggagccctgttggctgaagtgtagggcgtcctttctgattcctgaagtatatttattagccccacggcaaggaagaacagactcagaacgaagcccccgactccactcatcatcttgctctgagcagagtcagaccgtgccctccattctactgtgatagggcttgtctggctggggtgctccacttggcaagtgtagacctggcacca

SEQ ID NO: 12 - DNA, 454 bp, artificial sequence
  ESB marker
  misc_feature (435)..(446): N= A, T, C or G
gctgtccaaaaaggcctccgttatggaataattctttttattatctccgaagtactattctttaccggatttttctgagctttctaccactcaagcctcgcccccacccctgggggaggcggctgctgacccccaacaggcattcacccactaaaccccctagaagtcccactgctcaacacctctgtcctattggcttccggagtttctattacctgagcccatcatagtttaatagaaggggaccgaaagcatatattacaagccctatttatcaccatcacattaggagtctacttcacactactacaagcctcagaatactatgaagcaccttttactatctccgacggagtttacggctcaactttttttgtagccacaggcttccacggcctccacgtcatcattgggtccaacaaataacgcttctcnnnnnnnnnnnntgcagata

SEQ ID NO: 13 - DNA, 219 bp, artificial sequence
  ESB marker
  misc_feature (47)..(140): N = A, T, C or G
ggggaggtatctgtcacccacgcagaaatgcttctgacaggcggcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncttggccgaaaagcctgaggtagtctcggcggcagagcttccggcccagcttgtagtagaggcgccggcccacctaccc

SEQ ID NO: 14 - DNA, 386 bp, artificial sequence
  ESB marker
gaagcgttattggaggaggctaacctaggagcagaggatcagttcacgaagagcgagcgggtgaactcgacgtagtcaaaagcagtggggagttcgcggcccttgctgtccacgtagggcttcatgtgggagacgcagtagtcggcttgttcccgagtcaagttctggtacagctcctccttggtcacataaggcttcccttcagagctcacggcccggaaggcgctctcaatctcctcgctggacttgacgttctccgtctcacggctgatcataaaggacatgtactcttgcatggagacgtggacgtccctgttaggatccacagtgtccaagatggactcgaactcaaggtcgggctccccttcctccaacatgggcaggtc

SEQ ID NO: 15 - DNA, 472 bp, artificial sequence
  ESB marker
tctgcagaattcgcctttgagaagcgttatgggggcgaggtggtaaaggaagcttacaaaacaactattctttaaaaaaaaacaaaaaaacaaaaaaacaaaaaacagcaaaagccaaccggcccaattttgtctccagttttcaacgtgtgctttcgagcatttcagctgtttccagttactttagtttccagatattagtcttccatttagttttaagactaaatctcacttttggataaacacaaggaaatattttacttgctgaaaaatcactttactggataaagttacctcttatgcctttcagttttctaatccaactttctgacaaccagtggtaattaggaagttctaagttgcagttgtccctatgactttgggcttccctggtggctcagctggtcaaaaatctgcctgcaatgcgggagacctccaccccataacgcttctcaaaggcgaattctgcaga

SEQ ID NO: 16 - DNA, 424 bp, artificial sequence
  ESB marker
ttgagaagcgttattgtggggaggtcatagttgatgactaaggaaacttgctgtacatcaatacctctggccagtaggtcagtggtaatcaatactctgctggagccagagcggaactccctcatgataacgtctcgttctttttggtccatgtctccgtgcatggcagagacggtgaagtctcgggcatgcatcttctcggtgagccaatccaccttccttcgggtgttgatgaagatgactgcctgggtaatggtcagggtttcatacaagtcgcacagtgtgtccagcttccactcctctcgttccacattgatgtagaactgacggataccctccagcgtcaactcttccttcttgacaagaattctaattgggtccctcatgaacttcttggtcacctcccgcccataacgcttctcaa

SEQ ID NO: 17 - DNA, 474 bp, artificial sequence
  ESB marker
cttgtatggtgtatggaagttacttggtaaatccagaatcaggatacaatgtctccttgctatacgaccttgaaaatctgcctgcatccaaggattccatcgtgcatcaagctggcatgttgaaacgaaactgttttgcctctgtctttgagaaatacttccagttccaggaatgagggcaaggaatgagagttaggggcagttatccattatagggatgatgagaccatgtatgttgagtcaaaaaaagacagagtcacagtagtcttcagcacagtgtttaaggatgacgacgatgtggtcattggaaaggtgttcatgcaggagttcaaagaaggacgcagagccagccacacagccccacaggtcctcttcagccacagggaacctcccttagagctgaaagataccgatgccgccgtgggtgacaacattggctacattaccttcgtgctgttccctgccccaatataa

SEQ ID NO: 18 - DNA, 372 bp, artificial sequence
  ESB marker
  misc_feature (362)..(368): N = A, T, C or G
ccagtgtgttgcccctgagaagcgttatatgcggtagtgaggggaatttcaattacatcgagttcacacgcatccttaagcatggagcgaaagacaaagacgactaaaaagaacttcaaactccagccaaacgttccttgttgccactctgggtatttctgagactttctcttagagcctgttgcatgcccttagctttacagcttctgcctttcttttgtatttattctcagccatttggggcacatgcatctctataatcagactggatatgggacttcttgtcattttaagagtagaaaatagggtaatttaacttaccagctgccgtctaccctcccccaaagtcataacgcttctcnnnnnnncagc

SEQ ID NO: 19 - DNA, 535 bp, artificial sequence
  ESB marker
tctgcagaattcgcctctgagaagcgttatgctgagaggggggactggaagctttgctgatatttactcaatattcacaaggggcctgtgtaatgtgtttcacaggtagtgctaatgctcaatgcaagatgcatttcagccttgtaattcctttcatttgagtctttgaaccatgtccaatgaaccagagctcaaactaatcaattttgtagttggtatttgttggaggggaggcaggcatggacagcaatagggagtgagctggagagatgctttgctaaccatagtaaactgtgaaaaaatagttacttcctgaaaaaaggaaatattcttgagagcaccttcataatgtcatcaaatacatggctaaatacattgtcttgagcctccttcctaatgtttcttagttttttttcatattccatctttagtaattcaatttccccctctttttcctgcataatcttctcgcatgcttgagcacactccttttccactttttggatttccatttctaattgatcaatatatcttt

SEQ ID NO: 20 - DNA, 527 bp, artificial sequence
  ESB marker
agaagcgttatcgggtaggctaacctaggagcagaggatcagttcacgaagagcgagcgggtgaactcgacgtagtcaaaagcagtggggagttcgcggcccttgctgtccacgtagggcttcatgtgggagacgcagtagtcggcttgttcccgagtcaggttctggtacagctcctccttggtcacataaggcttcccttcagagctcagggcccggaaggcgctctcaatctcctcgctggacttgacgttctcggtctcacggctgatcataaaggccatgtactcttgcagggagacgtggccgtccctgttaggatccacagtgtccaggatggcctcgaactcagggtcgggctccccttcctccaccatgggcaggtcatagcccagggagcgcagacaggatttgaactcctggtggttcagccggccagacttgtccttgtcgaagtgtttgaacatcatgctgaattctttgagggccttacagataacgcttctcaaaggcgaattctgcagata

SEQ ID NO: 21 - DNA, 546 bp, artificial sequence
  ESB marker
gagaagcgttatggcggggaggtaccgaaagcacagtaatcactggtgtcgatattgtcatgagccatcacttgcaggaaaccagcttcacaaaagaagcctacaagaagtacatcaaagattacatgaagtcaatcaaagggaaacttgaagaacagagaccagaaagagtaaaaccttttatgacaggggctgcagaacaaatcaagcacatccttgctaatttcaaaaactatcagttctttattggtgaaaacatgaatccagatggcatggttgctctgctggactaccgtgaggatggtgtaaccccatatatgattttctttaaggatggtttagagatggaaaaatgttaacaaagttggcagttactttggatcaatcacctcccccccataacgcttctctaatgcttattcatgcagacaacaccaggacttagacagatgggactgatgtcatctcgagctcttcatttgttttgaacgttgatttatttggagcggaggcattgtttttgagaaaacgtgtcatgtaggtccc

SEQ ID NO: 22 - DNA, 310 bp, artificial sequence
  ESB marker
ggggtaggtcaaaaaaagtccaaaccaaaaacaaaacctgccaaaaccaacaaaaaacctccgaaatctgaagacaactgaatcaatccctgcagtctcactttctcttggaaagaaaagttggataatccaacccttttacaaaggataatacaagggtgacagttccaagctctcaggaacagggtcttagacgcttttggaggttgagaggcacaaaacggcagtctgaaaattcctttcatctcacggcactgattgagtttagacttgatttctcctcccctacctacccgatataacgcttctc

SEQ ID NO: 23 - DNA, 151 bp, artificial sequence
  ESB marker
gaagggcaggcgcgaaaggcagctacagccagtgagaaatcagatggcatttacacgggcctgagcacccggacccaggagacttatgagaccctgaagcatgagaaaccaccacaatagctttagaacagatgccctttgtcacttcctt

SEQ ID NO: 24 - DNA, 379 bp, artificial sequence
  ESB marker
aatgtaagggggattagagtgattatgggagcagctaaagatgagaggggctcagttttccgcaacactaaatctaaaaagtattttggcttcttactgtagagagcagacctctacaggaatcctacattggaaaagagacccagaggtctgcggttcactgctgccacactgtctcacatagtacctttggagtaggcctgacagagagcacagggaagcttcagaaacctgtaattcaagattttatttttttgagacgttctctctgatactgttccccgccagccttttttaaaagtttgagaaacttttcaagctctgcaaaaggggacaaagaatttgccttgcagtgtggggatatgattgagcggcagtg

SEQ ID NO: 25 - DNA, 251 bp, artificial sequence
  ESB marker
gtggtaggtgactgaggagtgtggcaagtttggtgctgtcaaccgtgtcatcatctaccaagagaagcagggcgaggaagaggacgcggagatcattgtcaagatttttgtggagttttccgtagcctctgagactcacaaggccatccaggccctcaatgggcgctggtttgctggccgcaaggtggtggctgaagtgtatgaccaggagcgttttgataacagtgacctctctgcatgacctccccccc

SEQ ID NO: 26 - DNA, 290 bp, artificial sequence
  ESB marker
gatcagtacagctgccgagtgaaacacgttactttggaacaaccccggatagttaagtgggatcgagacctgtaagcagcaccatcgagatttgaacattcttcatttggtataatatctggaaaattctgtttccctgctctttaatactgatatgcttttatgctttatgcgcataatcagaagtcatattcatgttaccataaataccttctttataattttaccgtgggtgctacatgtccatgtttgaccttcctaggcaggtgtctgcagtggaggtccacaaa

(19) United States
(12) Patent Application Publication
(10) Pub. No.: US 2007/0292850 A1
(43) Pub. Date: Dec. 20, 2007

(54) Identification of Diagnostic Markers for Communicable Subacute Spongiform Encephalopathies

(76) Inventors: Annelies Resink, Heverlee (BE); Magali Rouquette, Toulouse (FR)

Correspondence Address: NIXON & VANDERHYE, PC, 901 NORTH GLEBE ROAD, 11TH FLOOR, ARLINGTON, VA, 22203, US

(21) Appl. No.: 10/578,672

(22) PCT Filed: Nov. 10, 2004
(86) PCT No.: PCT/FR04/02892

§ 371 Date: May 9, 2006

(30) Foreign Application Priority Data
Nov. 13, 2003 (FR) 0313275
Dec. 10, 2003 (FR) 0314486

(51) Int. Cl. C12Q 1/68; G01N 33/53

(52) U.S. Cl. 435/6; 435/7.21

(57) ABSTRACT

The invention relates to biological markers of transmissible subacute spongiform encephalopathies and to uses thereof in diagnostic methods. The invention also relates to tools and/or kits that can be used for carrying out said methods (reagents, probes, primers, antibodies, chips, cells, etc.), to their preparation and to uses thereof. The invention can be used for detecting the presence of an infection in mammals including during the early phase.

The invention relates to biological markers of transmissible subacute spongiform encephalopathies and to uses thereof in diagnostic methods. The invention also relates to tools and/or kits that can be used for carrying out said methods (reagents, probes, primers, antibodies, chips, cells, etc.), to their preparation and to uses thereof. The invention can be used for detecting the presence of an infection in mammals including during the early phase.

Prion diseases, also called transmissible subacute spongiform encephalopathies or diseases due to non-conventional transmissible agents (NCTA), are central nervous system diseases that affect certain mammals, including humans.

The most well-known forms of said diseases are sheep scrapie in ovines, bovine spongiform encephalopathy (BSE) in bovines, Creutzfeld-Jakob disease (CJD), kuru and familial fatal insomnia in humans.

The infectious agent has not yet been conclusively determined, but the most widely accepted hypothesis is that these diseases are associated with the accumulation in brain of a prion protein (PrP) having an abnormal conformation relative to the conformation observed in healthy individuals.

The discovery of a new variant of CJD (vCJD) following the mad cow BSE epidemic in England confirms that said diseases are transmissible and can probably jump the species barrier through the feed. Their slow and fatal course is associated with lesions affecting solely the central nervous system.

With the discovery of vCJD, emergency measures were instituted to evaluate the extent of the BSE epidemic and to protect the public health. The European Union now requires routine testing of all meat from slaughtered bovines aged over 30 months. As the incubation period for BSE is about five years, during which time the infection can spread laterally and vertically, the development of a diagnostic test on live animals is of critical importance. An early test would offer a sure means to keep infected animals out of the food chain. The test currently in use only detects infected animals postmortem at a late stage of the disease.

There is an urgent need to develop a diagnostic test capable of detecting encephalopathies at an early stage in live animals and in a rapid manner. Such a test would make it possible to monitor all animals at risk by testing them many times throughout their lifetime.

The applicant's patent application WO02/074986 describes several genetic markers of encephalopathies. Through extensive research based on an innovative approach, different additional BSE markers have been identified and validated by hybridization experiments, enabling the development of a presymptomatic test that can be used on blood from a live mammal.

The identified markers were isolated by the DATAS method (patent application WO99/46403). DATAS identifies qualitative differences in gene expression and provides a systematic analysis of RNA splicing between two conditions: healthy/infected. DATAS leads to the identification of functionally distinct RNA variants. The DATAS method comprises three separate steps: collection of the tissue, isolation of RNA, and creation of a library containing qualitative differences and identifying novel gene fragments, which cannot be isolated by other genetic techniques.

By comparing qualitative gene expression in blood cells from healthy mammals and those infected naturally or experimentally with BSE, different signatures of genetic markers have been isolated. The naturally infected animals had terminal stage disease, whereas mammals infected by the oral route with 1 g of BSE-infected brain represent the early stage of the disease.

Implementation of the DATAS method on blood cells from cows led to the identification and isolation of several thousand genetic markers, divided into two libraries representing qualitative gene expression between healthy cows and naturally infected cows, on the one hand, and between healthy cows and experimentally infected cows, on the other hand.

The markers in these libraries were selected and validated by two approaches:

In the first approach, gene fragments common to the two DATAS libraries produced in this manner were identified. The sequences of these 11 markers are given in sequences SEQ ID NO: 16-26.

In the second approach, different clones from the two DATAS experiments were transferred to glass slides. The slides were hybridized with probes produced from biological material from naturally or experimentally infected cows and healthy cows used as controls. Through the use of two types of statistical analysis, SAM (Significance Analysis of Microarray) and PAM (Prediction Analysis of Microarray), comparing healthy versus infected animals, 15 clones were found to show a deregulation between healthy versus infected conditions. The 15 nucleic acid sequences are given hereinbelow as SEQ ID NO:1-15.

The invention thus provides a set of biological markers that can be used, alone or in combination(s), to detect, characterize or monitor a transmissible spongiform encephalopathy in a mammal. In particular, the invention can be used to detect the presence of prion diseases in mammalian subjects, particularly ovines, bovines and humans. The invention is particularly advantageous in that it can be carried out on live mammals, from biological fluids such as blood, plasma, platelets and the like.

One object of the invention relates to a method for detecting the presence or the risk of developing an encephalopathy in a mammal, comprising determining the presence (or the absence), in a biological sample from the mammal, of a target molecule selected in the group consisting of:
a) a nucleic acid comprising a sequence selected from SEQ ID NOs: 1-26 or a fragment thereof containing at least 5, preferably 6, 7, 8, 9 or 10 consecutive bases,
b) a nucleic acid having a sequence complementary to a sequence according to a),
c) a functional analogue of a nucleic acid according to a) or b), or
d) a polypeptide coded by a nucleic acid according to a) to c),
   the presence (or the absence) of said target molecule in the sample being an indication of the presence or the risk of developing an encephalopathy in said mammal.

In a particular variant, the method comprises determining (simultaneously) the presence or absence of at least 2, 3, 4, 5, 6, 7, 8, 9, 10 or more target molecules such as defined hereinabove. In fact, the invention makes it possible to establish and define a hybridization profile on a set of markers, in order to evaluate the presence or the risk of developing an encephalopathy in a mammal. The hybridization profile is typically produced by using a combination of several markers selected from among the aforementioned targets, for example containing the entire set of said targets.

The target molecule can be the complete sequence of the gene or RNA or protein corresponding to sequences SEQ ID NOs: 1-26, or a fragment thereof, for example a fragment containing a variable domain (splicing, deletion, polymorphism, etc.). A functional analogue more specifically denotes an analogue originating from another species (for example human, sheep, etc.), or a natural variant resulting for example from a polymorphism, splicing, etc.

In a particular embodiment, the method comprises determining the presence of at least one nucleic acid according to a) to c). Different methods by which to detect a nucleic acid species in a sample can be used in the invention, such as for example Northern Blot, selective hybridization, the use of supports coated with oligonucleotide probes, amplification of nucleic acid such as for example by RT-PCR, quantitative PCR and ligation-PCR, etc. Said methods can comprise the use of a nucleic probe (for example an oligonucleotide) capable of selectively or specifically detecting the target nucleic acid in the sample. The amplification can be carried out by different methods known to those skilled in the art, such as PCR, LCR, transcription-mediated amplification (TMA), strand displacement amplification (SDA), NASBA, the use of allele-specific oligonucleotides (ASO), allele specific amplification, Southern blot, single strand conformation analysis (SSCA), hybridization in situ (e.g., FISH), gel migration, heteroduplex analysis, and the like.

According to a preferred embodiment, the method comprises detecting the presence or the absence of a nucleic acid according to a) to c) by selective hybridization or selective amplification.

Selective hybridization is typically carried out by using nucleic probes; preferably immobilized on a support, such as a solid or semi-solid support having at least one surface, flat or not, on which nucleic probes can be immobilized. Examples of such supports are a slide, bead, membrane, filter, column, plate, and the like. They can be made of any compatible material, such as in particular glass, silica, plastic, fiber, metal, polymer, and the like. The nucleic probes can be any nucleic acid (DNA, RNA, PNA, etc.), preferably single-stranded, comprising a sequence specific of a target molecule such as defined in a) to c) hereinabove. The probes typically comprise from 5 to 400 bases, preferably from 8 to 200, more preferably fewer than 100. The probes can be synthetic oligonucleotides, produced on the basis of the sequences of the target molecules of the invention according to conventional synthetic methods. The probes can also be synthesized directly in situ, on the support, according to methods known to those skilled in the art. The probes can also be produced by genetic methods, for example by amplification, recombination, ligation, and the like. Said probe is another object of the invention, as is the use thereof (mainly in vitro) for detecting an encephalopathy in a subject. In an especially preferred manner, a set of nucleic probes is used comprising all or a fragment of at least 5 consecutive bases of each one of sequences SEQ ID NO: 1-26, or a strand complementary to same, advantageously immobilized on a support.

The hybridization can be carried out in classical conditions, known to those skilled in the art and adjustable by same (Sambrook et al). In particular, the hybridization can be carried out under conditions of high, intermediate or low stringency, depending on the desired level of sensitivity, the amount of available material, etc. For example, suitable hybridization conditions include a temperature comprised between 62 and 67° C. for 2 to 18 hours. After hybridization, different washes can be carried out to eliminate unhybridized molecules, typically in SSC buffers containing SDS, such as a buffer containing 0.1 to 10× SSC and 0.1% SDS.

In a typical embodiment, the nucleic acids (or chips or supports) are prehybridized in hybridization buffer (Rapid Hybrid Buffer, Amersham) typically containing 100 μg/ml of salmon sperm DNA at 65° C. for 30 min. The nucleic acids of the sample are then contacted with the probes (typically deposited on the support or chip) at 65° C. for 2 to 18 hours. Preferably, the nucleic acids of the sample are labelled beforehand, by any known labelling (radioactive, enzymatic, fluorescent, luminescent, etc.). The supports are then washed in 5× SSC, 0.1% SDS buffer at 65° C. for 30 min, then in 0.2× SSC, 0.1% SDS buffer. The hybridization profile is analyzed according to conventional methods, such as for example by measuring the labelling of the support by means of suitable instrumentation (for example. InstantImager, Packard Instruments). The hybridization conditions can naturally be adjusted by those skilled in the art, for example by modifying the hybridization temperature and/or the salt concentration of the buffer.

Selective amplification is preferably carried out by using a primer or a primer pair allowing the amplification of all or part of one of the target nucleic acids in the sample, when it is present therein. The primer can be specific of a target sequence according to SEQ ID NO: 1-26, or of a region flanking the target sequence in a nucleic acid of the sample. The primer typically contains a single strand nucleic acid, having a length advantageously comprised between 5 and 50 bases, preferably between 5 and 30. Said primer is another object of the invention, as is the use thereof (mainly in vitro) for detecting an encephalopathy in a subject.

In another embodiment, the method comprises determining the presence of a polypeptide according to d). Detection of a polypeptide in a sample can be carried out by any known method, such as in particular by means of a specific ligand, for example an antibody or a fragment or derivative of same. Preferably, the ligand is an antibody specific of the polypeptide, or a fragment of said antibody (for example a Fab, Fab′, CDR, etc.), or a derivative of said antibody (for example a single chain antibody, ScFv). The ligand is typically immobilized on a support, such as a slide, bead, column, plate, and the like. The presence of the target polypeptide in the sample can be detected by visualizing a complex between the target and the ligand, for example by using a labelled ligand, by using a second labelled visualization ligand, etc. Well-known immunologic methods that can be used are ELISA, RIA, and the like.

Antibodies specific of target polypeptides can be produced by conventional methods, in particular by immunizing a non-human animal with an immunogen comprising the polypeptide (or an immunogenic fragment of same), and recovering the antibodies (polyclonal) or producing cells (to produce monoclonal antibodies). Methods to produce poly- or mono-clonal antibodies, ScFv fragments, human or humanized antibodies are described for example in Harlow et al., Antibodies: A Laboratory Manual, CSH Press, 1988; Ward et al., Nature 341 (1989) 544; Bird et al., Science 242 (1988) 423; WO94/02602; U.S. Pat. No. 5,223,409; U.S. Pat. No. 5,877,293; WO93/01288. The immunogen can be produced by synthesis, or by expression of a target nucleic acid such as defined hereinabove, in a suitable host. Said monoclonal or polyclonal antibody, and derivatives thereof having the same antigenic specificity, are also an object of the invention, as is the use thereof for detecting an encephalopathy.

The inventive method can be used with any biological sample from the mammal being tested, in particular any sample containing nucleic acids or polypeptides. Advantageously the sample is blood, plasma, platelets, saliva, urine, stool, etc., more generally it is any tissue, organ or, advantageously, biological fluid containing nucleic acids or polypeptides. In a preferred embodiment, the sample is a sample of blood or plasma. The sample can be obtained by any known method, for example by non-invasive sampling, from sample collections or libraries, and the like. The sample can also be pretreated to facilitate the accessibility of the target molecules, for example by lysis (mechanical, chemical, enzymatic, etc.), purification, centrifugation, separation, and the like. The sample can also be labelled, to facilitate detecting the presence of the target molecules (fluorescent, radioactive, luminescent, chemical, enzymatic labelling, etc.).

The invention can be used in any mammal, preferably selected in the group consisting of bovines, ovines and humans. The inventive method is particularly useful for detecting sheep scrapie in ovines, Bovine Spongiform Encephalopathy (BSE) in bovines, and Creutzfeld-Jakob disease (CJD), kuru and familial fatal insomnia in humans.

A particular object of the invention relates to a method for detecting the presence or the risk of developing BSE in a bovine, comprising determining the presence (or the absence), in a biological sample from the bovine, of one or more target molecules selected in the group consisting of:
a) a nucleic acid comprising a sequence selected from SEQ -ID NO: 1-26 or a fragment thereof containing at least 5, preferably 6, 7, 8, 9 or 10 consecutive bases,
b) a nucleic acid having a sequence complementary to a sequence -according to a),
c) a polypeptide coded by a nucleic acid according to a) or b).

Another particular object of the invention relates to a method for detecting the presence or the risk of developing BSE in a bovine or ovine, comprising contacting a biological sample from the bovine or ovine containing nucleic acids with a product comprising a support on which is immobilized at least one nucleic acid comprising a sequence selected from SEQ ID NO: 1-26, a fragment thereof containing at least 5 consecutive bases, or a nucleic acid having a sequence complementary thereto, and determining the hybridization profile, said profile indicating the presence or the risk of developing BSE in the bovine or ovine. Preferably, the product comprises at least 2, 3, 4, 5, 6, 7, 8, 9, 10 different nucleic acids selected from the aforementioned nucleic acids. In a particular embodiment, the product comprises each of the nucleic acids of sequence SEQ ID NO: 1-26, a fragment thereof containing at least 5 consecutive bases, or a nucleic acid having a sequence complementary thereto.

Another object of the invention relates to a product comprising a support on which is immobilized at least one nucleic acid comprising a sequence selected from SEQ ID NO: 1-26, a fragment thereof containing at least 5 consecutive bases, a nucleic acid having a sequence complementary thereto or a functional analogue thereof. Preferably, the product comprises at least 2, 3, 4, 5, 6, 7, 8, 9, 10 different nucleic acids selected from the aforementioned nucleic acids. In a particular embodiment, the product comprises each of the nucleic acids of sequence SEQ ID NO: 1-26, a fragment thereof containing at least 5 consecutive bases, or a nucleic acid having a sequence complementary thereto.

Another object of the invention relates to a product comprising a support on which is immobilized at least one polypeptide coded by a nucleic acid comprising a sequence selected from SEQ ID NO: 1-26, a fragment thereof containing at least 15 consecutive bases, a nucleic acid having a sequence complementary thereto or a functional analogue thereof. Preferably, the product comprises at least 2, 3, 4, 5, 6, 7, 8, 9, 10 different polypeptides selected from the aforementioned polypeptides.

The support can be any solid or semi-solid support having at least one surface, flat or not, on which to immobilize the nucleic acids or polypeptides. Examples of such supports are a slide, bead, membrane, filter, column, plate, and the like. They can be made of any compatible material, such as in particular glass, silica, plastic, fiber, metal, polymer, polystyrene, teflon, and the like. The reagents can be immobilized on the surface of the support by known methods or, in the case of nucleic acids, synthesized directly on the support. Immobilization techniques include passive adsorption (Inouye et al., J. Clin. Microbiol. 28 (1990) 1469), covalent binding. Techniques are described for example in WO90/03382, WO99/46403. The reagents immobilized on the support can be arranged in a predefined order, to facilitate the detection and identification of the complexes formed, and at a variable and adjustable density.

In one embodiment, the product of the invention comprises a multiplicity of synthetic oligonucleotides, having a length comprised between 5 and 100 bases, specific of one or more target nucleic acids defined in a) to c).

The inventive products typically comprise control molecules, allowing to calibrate and/or normalize the results.

Another object of the invention relates to a kit comprising a compartment or container containing at least one nucleic acid comprising a sequence selected from SEQ ID NO: 1-26, a fragment thereof containing at least 5 consecutive bases, a nucleic acid having a sequence complementary thereto or a functional analogue thereof. Preferably, the product comprises at least 2, 3, 4, 5, 6, 7, 8, 9, 10 different nucleic acids selected from the aforementioned nucleic acids. In a particular embodiment, the product comprises each of the nucleic acids of sequence SEQ ID NO: 1-26, a fragment thereof containing at least 5 consecutive bases, or a nucleic acid having a sequence complementary thereto. The kit can also comprise reagents for a hybridization or immunologic reaction and, as the case may be, controls and/or instructions.

Another object of the invention relates to the use of a product or kit such as defined hereinabove for detecting an encephalopathy in a mammalian subject.

Another object of the invention relates to a nucleic acid comprising a sequence selected from SEQ ID NO: 1-26, or a fragment thereof containing at least 5 consecutive bases, or a nucleic acid having a sequence complementary thereto, or a functional analogue thereof, in particular an analogue originating from another species. The invention also relates to a cloning or expression vector containing said nucleic acids, and any recombinant cell comprising said vector or nucleic acid.

Another object of the invention relates to the use of a nucleic acid comprising a sequence selected from SEQ ID NO: 1-26, or a fragment thereof containing at least 5 consecutive bases, or a nucleic acid having a sequence complementary thereto, or a functional analogue thereof, in particular an analogue originating from another species, for detecting (mainly in vitro) an encephalopathy in a mammalian subject.

According to a particular example of an inventive embodiment, a blood sample is taken from a mammal to be tested. The blood sample is treated so as to render the nucleic acids more accessible, and said nucleic acids are labelled. The nucleic acids are then deposited on a product such as defined hereinabove and the hybridization profile is determined, allowing to diagnose the presence or absence of an encephalopathy in the subject. The inventive method is simple, carried out ex vivo, on live animals, and enables early detection of a prion disease.

It is understood that any equivalent method can be used in the scope of the invention for determining the presence of a target molecule. The list of sequences is given hereinbelow (N denotes any base).

1-12. (canceled)

13. A method for detecting the presence or the risk of developing an encaphalopathy in a mammal, comprising determining the presence, in a biological sample from the mammal, of a target molecule selected in the group consisting of:
a) a nucleic acid comprising a sequence selected from SEQ ID NO: 1-26 or a fragment thereof containing at least 5, preferably 6, 7, 8, 9 or 10 consecutive bases,
b) a nucleic acid having a sequence complementary to a sequence according to a),
c) a functional analogue of a nucleic acid according to a) or b) originating from another species or a natural variant, or
d) a polypeptide coded by a nucleic acid according to a) to c),
the presence of said target molecule in the sample being an indication of the presence or the risk of developing an encephalopathy in said mammal.

14. Method according to claim 13, comprising determining the presence of at least 2, 3, 4, 5, 6, 7, 8, 9, 10 or more target molecules.

15. Method according to claim 13, comprising detecting the presence or the absence of a nucleic acid according to a) to c) by selective hybridization or selective amplification.

16. Method according to claim 13, comprising detecting the presence or the absence of a polypeptide according to d) by means of a specific antibody or a fragment or derivative of same.

17. Method according to claim 13, for detecting the presence or the risk of developing BSE in a bovine, comprising determining the presence, in a biological sample from the bovine, of one or more target molecules selected in the group consisting of:
a) a nucleic acid comprising a sequence selected from SEQ ID NO: 1-26 or a fragment thereof containing at least 5, preferably 6, 7, 8, 9 or 10 consecutive bases,
b) a nucleic acid having a sequence complementary to a sequence according to a),
c) a polypeptide coded by a nucleic acid according to a) or b).

18. Method according to claim 13, for detecting the presence or the risk of developing BSE in a bovine or ovine, comprising contacting a biological sample from the bovine or ovine containing nucleic acids with a product comprising a support on which is immobilized at least one nucleic acid comprising a sequence selected from SEQ ID NO: 1-26, a fragment thereof containing at least 5 consecutive bases, or a nucleic acid having a sequence complementary thereto, and determining the hybridization profile, the profile indicating the presence or the risk of developing BSE in the bovine or ovine.

19. Method according to claim 13, for detecting the presence or the risk of developing BSE in a bovine or ovine, comprising contacting a biological sample from the bovine or ovine containing nucleic acids with a product comprising a primer allowing amplification of all or part of a target nucleic acid such as defined in claim 13, said primer being single-stranded and having a length comprised between 5 and 50 bases, the presence of amplification products being an indication of the presence or the risk of developing BSE in the bovine or ovine.

20. The method of claim 13, wherein the sample is a blood sample.

21. A product comprising a support on which is immobilized at least one nucleic acid comprising a sequence selected from SEQ ID NO: 1-26 or a fragment thereof containing at least 5, preferably 6, 7, 8, 9 or 10 consecutive bases, or a sequence complementary thereto.

22. A product comprising a support on which is immobilized at least one polypeptide coded by a nucleic acid comprising a sequence selected from SEQ ID NO: 1-26 or a fragment thereof containing at least 9 consecutive bases, or a sequence complementary thereto.

23. A kit comprising a compartment or container containing at least one nucleic acid comprising a sequence selected from SEQ ID NO: 1-26 or a fragment thereof containing at least 5, preferably 6, 7, 8, 9 or 10 consecutive bases, or a sequence complementary thereto.
